(19) United States
(12) Patent Application Publication
(10) Pub. No.: US 2024/0150247 A1
(43) Pub. Date: May 9, 2024

(54) CONTROL PROCESS FOR CONTROLLING CALCINATION OF CLAYS FOR THE CEMENT INDUSTRY

(71) Applicants: thyssenkrupp Polysius GmbH, Beckum (DE); thyssenkrupp AG, Essen (DE)

(72) Inventors: Michael ENDERS, Muenster (DE); Guido GRUND, Werl (DE)

(73) Assignees: thyssenkrupp Polysius GmbH, Beckum (DE); thyssenkrupp AG, Essen (DE)

(21) Appl. No.: 18/284,048

(22) PCT Filed: Mar. 15, 2022
(86) PCT No.: PCT/EP2022/056594

§ 371 Date: Sep. 25, 2023

(30) Foreign Application Priority Data
Mar. 26, 2021 (BE) BE 2021/5231
Mar. 26, 2021 (DE) 10 2021 203 044.0

(51) Int. Cl. C04B 33/32; C04B 33/04; G01N 33/24

(52) U.S. Cl. CPC C04B 33/32; C04B 33/04; G01N 33/24

(57) ABSTRACT

A control process for controlling a production process for calcined clays with a calciner including, capture of at least one temperature in the calciner, taking of at least one sample of the calcined clay, production of a reproducible size distribution of the sample, adjustment of the sample mass, conditioning of the sample to a first measuring temperature and of an alkali to a first measuring temperature, combining of the sample with an alkali, temporal capture of the energy generated by the sample-alkali mixture, quantitative evaluation of the time-energy profile captured for the first exothermic reaction and determination of the amount of energy released by the sample, correlation of the amount of energy captured with the temperature and residence time captured, and comparison with previously captured combinations, temperature and residence time values.

The present invention relates to a control process for optimizing product quality, in terms of the reactivity of the end product, in the calcining of clays.

To get a grip on the problem of CO₂ emissions, the present-day plan is to replace scarce composite materials such as slag sand and fly ash by calcined clay as a constituent in cements.

EP 3 218 320 B1 discloses a process for the heat treatment of natural clays and/or zeolites, by calcining the clay and/or the zeolite in the calcining zone in an entrained flow calciner or a fluidized bed in a temperature range from 350° C. to 1050° C. under reducing conditions, where the calcination under reducing conditions is accompanied at least by partial reduction of trivalent iron, causing reddish coloration, to divalent iron, more particularly to magnetite (FeO·Fe₂O₃).

U.S. Pat. No. 9,458,059 B2 discloses a process for producing synthetic pozzolans having desired color characteristics, with cooling under reducing conditions.

DE 10 2011 014 498 B4 discloses a process for producing a clinker substitute for use in cement production, where the clay is heat-treated under reducing conditions at a temperature of 600° C. to 1000° C. and the reduction product undergoes intermediate cooling in the absence of oxygen to a temperature<300° C.

DE 10 2008 020 600 B4 discloses a process for the heat treatment of fine mineral solids, where the solids are passed through a flash reactor in which they are brought into contact with hot gases at a temperature of 450° C. to 1500° C. and a residence time of between 0.5 and 20 seconds, and where the solids are subsequently guided at a temperature of 500° C. to 890° C. through a residence time reactor, from which they are taken off after a residence time of 1 to 600 minutes.

US 2012/160 135 A1 discloses a process for producing synthetic pozzolans having desired color properties, with cooling under a reducing atmosphere.

EP 3 615 489 A2 discloses a process for producing gray synthetic pozzolans, with a first rapid cooling below 600° C.

WO 2015/039 198 A1 discloses a process for producing a preparation for partial substitution of Portland cement with gray color.

WO 2016/041 717 A1 discloses the determination of the reactivity of a product.

Whereas the reaction of lime (roughly speaking, CaCO₃) to give cement clinker (roughly speaking, CaO) generates one mole of CO₂ for each mole of CaO, the calcining of clays releases only water. Here, the silicates/aluminates in the clay are reacted and converted from a nonreactive form into a reactive form, in which the silicates/aluminates, after reaction in a basic medium, are initially soluble and are able to react further with the calcium released in the reaction of clinker, to give calcium aluminum silicate hydrate phases (CASH). If these substances, however, are heated further or longer, they undergo transition into a glassy state, and at further-increased temperatures into inert mineral phases (spinels, mullite), and so lose their reactive condition again.

Considering, purely illustratively, the calcining of a theoretical clay at different temperatures, with a constant residence time, it is found that a first increase in the reactivity is posted only at a temperature above, for example, 500° C., and is boosted significantly above 600° C. In the range from 700° C. to 850° C., for example, an incipient plateau with maximum reactivity is developed. At temperatures above 900° C., for example, a rapid fall in the reactivity is then posted. It is therefore desirable for calcining to be carried out as far as possible in the region of this plateau (usually at the lower end, for reasons of cost).

The reactive properties, in binders, of clays and of the products which can be produced from them by calcining, however, are very difficult to characterize analytically. Chemical and mineralogical analyses permit only indirect conclusions regarding the reactivity, via analogies. Effective identification and quantification are possible for highly crystalline constituents such as quartz, by means of X-ray diffraction; however, these constituents are the constituents which are not reactive. Calcined clays are amorphous and therefore cannot be adequately quantified by X-ray diffraction. Attempts are therefore sometimes made to obtain information on the composition by means of NIR spectroscopy, but even this only provides inadequate and insufficiently quantitative information regarding the reactivity of the calcined clays.

There are, though, methods of measurement which are able to provide effective determination of the reactivity of calcined clays, as known, for example, from F. Avet et al., Development of a new rapid, relevant and reliable (R³) test method to evaluate the puzzolanic reactivity of calcined kaolinitic clay, Cement and Concrete Research 85 (2016) 1-11, Elsevier. This method is designed, as in section 3 Methods, subsection 3.1, for a measurement time of 7 days. Active regulation of a production plant is impossible with a period of this kind.

WO 2016/096 911 A1 discloses a calorimetric measuring facility for determining the reactivity.

Roger S. Almenares et al. in “Industrial calcination of kaolinitic clays to make reactive pozzolans”, Case Studies In Construction Materials, vol. 6, 1 Jun. 2017 (2017-06-01), pages 225-232, XP055554255, ISSN: 2214-5095, DOI: 10.1016/j.cscm.2017.03.005 disclose the industrial calcining of kaolinitic clay for producing reactive pozzolans.

WO 01/32581 A1 discloses a checking system for a clinker plant.

It is an object of the invention to provide innovative control that is aimed not at different physical parameters but instead at the actual reactivity and can be used for prompt feedback and hence active control.

This object is achieved by the control process having the features specified in claim 1. Advantageous developments are evident from the dependent claims, the description hereinafter, and the drawings.

The control process of the invention is used for controlling a production process for calcined clays with a calciner. Processes of this kind are known extensively from the prior art. The control process is independent of the specific production process and can be applied to all corresponding process variants for the production of calcined clays. The control process comprises the following steps:
a) capture of at least one temperature in the calciner and optionally of a first mean residence time of the clay in the calciner,
b) taking of at least one sample of the calcined clay,
c) production of a reproducible size distribution of the sample,
d) adjustment of the sample mass to a predefined sample mass,
e) conditioning of the sample to a first measuring temperature,
f) combining of the sample with an alkali, which is preferably likewise conditioned to a first measuring temperature,
g) temporal capture of the energy generated by the sample-alkali mixture at constant first measuring temperature for a first period,
h) quantitative evaluation of the time-energy profile captured in step g) for the first exothermic reaction and determination of the amount of energy released by the sample for the first exothermic reaction,
i) correlation of the amount of energy captured in step h) with the temperature and residence time captured in step a), and comparison with previously captured combinations of amount of energy, temperature and residence time values,
j) active control of the temperature and/or of the residence time in the calciner in the direction of increasing the amount of energy anticipated for a further sample.

In step a), the temperature in the calciner is captured. Because the temperature in the calciner is different at different points, it is particularly essential for the process that the temperature is always measured in the same way, i.e., for example, with the same sensor at the same point. Additionally, of course, it is also possible to capture further temperatures at further points and to consider them in order to produce a more exact picture, if, for example, different temperature profiles would result from the use of very different fuel having very different combustion behavior.

In step a), moreover, it is also possible to capture the mean residence time, if it can be controlled in a targeted way, especially separately from the temperature in the calciner. If the residence time is not adjustable, and in particular cannot be altered or can be altered only by a change in temperature, then there is no need for additional capture of the residence time as well as the temperature in the calciner.

The taking of the sample of the calcined clay in step b) may preferably take place automatically. The automated sampling is preferably connected to an automated sample preparation. The sample taken may be significantly greater than the predefined sample mass, so as to allow a retention sample as well to be taken at the same time from the sample taken for the purpose of implementing the process of the invention.

The SI unit for the mass is the kilogram (kg). The sample mass, however, may be measured preferably in grams (g) or milligrams (mg). Of course, especially in regions of Anglo-Saxon influence, for example, different units are possible, such as pounds and ounces. The skilled person is capable of converting between these units.

The adjustment of the sample mass to a predefined sample mass in step d) is accomplished by reducing the amount of the sample to a mandated size. Since losses occur in step c), for example, the sample taken in step b) will be larger than the sample used in steps e), f) and g). If, for example, a sample of exactly 10.0 g (illustrative mandated size) is taken for this purpose, it is possible in step b), for example, to take a sample of approximately 25 g, which is ground in step c), for example. In step d), then, a sub-sample of precisely 10.0 g is taken from this sample, and the rest of the sample is discarded. The mandated size is therefore dependent solely on the requirements of the apparatus used for step g).

It is essential that the only peak captured in step h) is the peak known as the initial peak, the first measurable exothermic reaction. Contemplating the heat given off by the sample over time, it is found that one or more exothermic reactions take place in succession, and there are therefore two or more maxima in the plot of energy against time. While the further steps (second and all further maxima) take place over a period of up to 7 days and provide an exact picture of the reactivity and the setting behavior, the invention contemplates only this first reaction step (the first maximum in the plot of the heat given off as a function of the time). It has emerged that the amount of heat released here, determined for example as the peak maximum, integral, area under the curve, or cumulative heat given off, has a very good correlation to the reactivity of the calcined clay. Hitherto, no attention was generally paid to this region, since firstly a rapid manual sample preparation procedure lasts for several minutes, and secondly the opening of the measuring cell introduces a thermal disruption, which necessitates verification of the so-called baseline. These two effects, through variation in time and intensity, disrupt the initial region of the measurement, which already comprises the initial peak. It is therefore essential to the invention that a size distribution is established which is comparable for all the samples. Less relevant in this context is the adjustment of a defined average size or a defined breadth of the size distribution, and nor is it necessary to know the size distribution exactly. It is enough merely to be able to achieve continuity over all the measurements. A reproducible size distribution of the sample is produced in step c) preferably by grinding. It has proven to be the case that if preparation is always the same, in the same mill for the same time, a sufficiently uniform size distribution is produced even when there are certain fluctuations in the original sample. With particular preference, therefore, sample preparation takes place automatically, since this allows the uniformity to be increased further.

The use of the initial peak, in accordance with the invention, actually enables only the control process of the invention. Between step b) and step j), of course, a certain time elapses, typically of one to two hours. This of course means that the control only takes place with a time offset. Since, however, production is typically operated continuously and the fluctuations are usually fairly small and/or long-term, because, for example, a raw material from a different stockpile is supplied and has somewhat different properties, it is nevertheless possible to carry out sufficiently prompt control on the production timescale. The benefit of the invention may therefore be realized even if, as a result of the process, there is a corresponding difference between sampling in step b) and control in step j). The great advantage relative to the conventional processes therefore lies in a time gain from previously two to seven days to only two hours as a result of the restriction to the initial peak rather than the entire setting behavior. The prior art, indeed, teaches that the initial peak specifically is not considered, as it is usually more greatly influenced by sample preparation and the like than are the later events in the setting behavior. The fact that, nevertheless, the initial peak is representative of the overall setting behavior, if sample preparation is carried out in the manner of the invention, is the new feature in this control process.

It has also proven very advantageous to adjust the sample to the first measuring temperature. As well as in step e), this may also take place earlier. During a grinding event, however, the amount of energy introduced is generally comparatively large, causing the sample to heat up. With particular preference, the alkali added in step f) is also conditioned to exactly the first measuring temperature even before the addition. The closer the sample-alkali mixture introduced into the measurement is to the first measuring temperature, the more exact the results of measurement are for the initial peak which is captured in step g) and evaluated in step h). The first measuring temperature is adjusted preferably with an accuracy of 0.001 K to 0.3 K, more preferably with an accuracy of 0.001 K to 0.1 K, with further preference of 0.01 K to 0.1 K.

With particular preference, measurement is commenced as soon as possible after the addition of the alkali to the sample. This is facilitated firstly if both components already have the same first measuring temperature before being brought together. Secondly, the combination in step f) takes place with as little energy input as possible—for example, by means of ultrasound or of an intensive mixer based on compressed air. Brief, intimate mixing is preferred, allowing the sample to be subsequently measured without further conditioning times, since the reaction of the calcined clay with the alkali begins directly upon the addition. Electrical or mechanical mixing apparatuses are suitable only if it is possible to ensure introduction of heat at well below the heat released during the reaction.

As a result of this focusing on homogenization and thermal stabilization of the sample prior to the addition of alkali and the start of measurement, the initial peak can already be meaningfully evaluated.

In step i), a correlation takes place, to obtain therefrom a control statement for the active control in step j). In this case, a correlation is necessary between the amount of energy determined in step h) and the temperature captured in step a), since the reactivity and hence the amount of energy captured as a result of the initial peak achieves a maximum at the optimum, rather than having a monotonally ascending or falling dependence on the temperature. If, in comparison with previous measurement values, a falling amount of energy was then found, and hence a drop in the reactivity of the product, this information on its own would not yet reveal whether the temperature has to be raised or lowered in order to regain the optimum. That is only evident from the correlation with the respective temperature values. A complicating factor is that the reactants, the clays that are used, are also different, and so it may be found in a subsequent cycle, following a correction, that the correction should have gone in the other direction, with correction then taking place accordingly. It is not automatically necessary here to control to the maximum, since the maximum is part of a plateau, and so control is aimed more at ranging within the plateau. For the control here, it is also possible to employ the energy requirement of the plant, so that production takes place more toward the lower (colder) end of the plateau in order to reduce the heating costs and the associated emissions, for example. It is also possible to employ further parameters as additional control criteria for optimization within the plateau.

In a further embodiment of the invention, the alkali selected in step f) comprises an alkali metal hydroxide solution having a pH of between 9 and 15, preferably between 10.25 and 15, more preferably between 12 and 14.6. With particular preference, the alkali is an aqueous sodium hydroxide solution having a concentration of between 0.1 mol/l and 1 mol/l. For example, an aqueous sodium hydroxide solution having a concentration of 1 mol/l is used (pH˜14).

In a further embodiment of the invention, the mass of alkali added is 0.5 times to 5 times, preferably 1 times to 5 times, more preferably 2 times to 3 times the mass of the sample. For a given mass, therefore, there is a weight ratio of 1:1 for sample to alkali and, at 5 times the mass of alkali, a weight ratio of 1:5 for sample to alkali. Where, for example, 5 g is used as the predefined sample mass, 5 to 15 g of a for example 1 mol/l aqueous sodium hydroxide solution—10 g, for example—are added to the sample and combined with it. A first effect of this is to provide a sufficient amount of hydroxyl ions. Furthermore, there is also sufficient volume available for complete wetting of the sample surface and also for filling of all the pores. The amount of alkali added is preferably adapted automatically in the same ratio to minor by any differences in the introduced weight to which, for an amounts of sample that are found to deviate from the target value. Accordingly, a constant ratio between sample and alkali is ensured. This also facilitates subsequent standardization of the initial peak energy obtained to the exact weight introduced.

In a further embodiment of the invention, the alkali added in step f) comprises an alkali former and water, where the alkali former and the water in reaction with one another generate a solution having a pH of between 9 and 15, preferably between 10.25 and 15, more preferably between 12 and 14.6. For example and in particular, the alkali former is selected from the group encompassing alkali metal hydroxide, alkali metal oxide, alkaline earth metal hydroxide, alkaline earth metal oxide, and substances, mixtures or compositions which comprise them or release them on reaction with water. The alkaline earth metal oxide may more particularly be calcium oxide, particular preference being given to a composition which comprises or releases calcium oxide, a cement clinker. For example, a sample of 2.5 g of calcined clay can be mixed with 2.5 g of cement clinker and 5 g of water. As a result, the reactivity of the calcined clay can be studied in a particularly realistic environment.

In a further embodiment of the invention, steps c) to g) take place automatically in an environment acclimatized to the first measuring temperature. This allows the sample to be thermally stabilized relatively quickly.

In a further embodiment of the invention, the sample mass is adjusted to the mandated sample mass in step d) to an accuracy of at least 2%, preferably at least 0.5%, more preferably at least 0.1%, very preferably to at least 0.02%. The greater the accuracy of the adjustment of the sample mass to the mandated sample mass, the smaller the measurement error for the amount of energy released in the initial peak. If the amount of alkali added is adapted to the precise weight introduced, then subsequent standardization is likewise easier to accomplish.

In accordance with the invention, the quantitative evaluation of the time-energy profile captured in step g) for the first exothermic reaction takes place for the period from the addition of the alkali and up to minute 120, preferably from 30 seconds after the addition of the alkali up to minute 120, more preferably from the first minute after the addition of the alkali up to minute 120, more preferably from the second minute after the addition of the alkali up to minute 70. The period from the addition of the initiating liquid up to the start of measurement may be employed as a null value. Preferably, therefore, only the first 30 seconds up to a maximum of two minutes are not considered, as this covers the time needed for sample mixing and for the transport of the sample mixed with the alkali into the analytical instrument up to the first measurement of a temperature change in the sample-alkali mixture. Accordingly, rather than the full reaction time of around 7 days, only the period from around an hour up to two hours after addition of the alkali is contemplated. This also enables prompt feedback to the production process. This time window has proven meaningful and yet sufficiently short. It would of course be advantageous if addition of alkali and mixing event could take place within the capturing, allowing the immediate start—i.e., the period of the first two minutes—to be captured as well. As represented in FIG. 2, the initial peak is typically already falling after 10 to 20 min. Because of the sharp drop, there is no longer any significant increased value obtained after just over two hours, preferably after just one hour; the area measured by integration under the measurement curve no longer increases significantly, and often, indeed, due to the noise, it is imperceptible. Because of the shortening of the measuring time, however, the feedback can be improved; for this, a slight deterioration in the evaluation due to any incomplete capture of the last end of the initial peak is readily acceptable and, indeed, is preferred.

In a further embodiment of the invention, the first measuring temperature selected is in the range from 20° C. to 40° C. For example and preferably, in Europe, 20° C., 21° C. or 22° C. is selected, while in hotter regions, such as the subtropics, a temperature of 27° C. is selected, for example. A higher temperature, 38° C. to 40° C. for example, may be selected so as to increase the reaction rate and hence shorten the time interval for the initial peak. A higher temperature is possible, but requires a greater heating effort and hence ongoing costs.

In a further embodiment of the invention, as well as the first measuring temperature, the process is carried out in alternation or in parallel with a second measuring temperature. For example, 20° C. is selected as the first measuring temperature and 40° C. as the second measuring temperature. This allows additional information to be determined, via the thermal effect of the temperature on the reactivity, about the activation energy.

In a further embodiment of the invention, the predefined sample mass selected is between 1 g and 200 g, preferably between 2 g and 20 g.

In a further embodiment of the invention, step a) additionally captures the reactant batches from which and the mixing ratio in which the clay is supplied to the calcination. For example, a reactant batch may be a stockpile or a similar storage region containing, generally, unitary material. In step i), the information as to the reactant batches from which and the mixing ratio in which the clay has been supplied to the calcination is additionally used. Consideration is given in particular to which reactant batches, in the case of a higher mixing fraction, lead to higher or lower reactivity. As a result, it is possible to produce the desired target reactivity for the product using the lowest reactant reactivities and hence the most favorable starting material. For control in step j), therefore, consideration is given additionally to the selection and the mixing ratio between the reactant batches.

In a further embodiment of the invention, the control process, additionally to the characterization of each product batch, comprises a batch characterization process, the batch characterization process being carried out with the following steps:
A) heating of a batch sample to a batch temperature of 600° C. to 1000° C., preferably 600° C. to 950° C., for a batch time of 1 s to 60 min, preferably of 30 s to 20 min, more preferably of 30 s to 5 min,
B) production of a reproducible size distribution of the batch sample,
C) adjustment of the batch sample mass to a predefined sample mass,
D) conditioning of the batch sample to a first measuring temperature,
E) combination of the batch sample with an alkali,
F) temporal capture of the energy generated by the batch sample-alkali mixture at constant first measuring temperature for a first period,
G) quantitative evaluation of the time-energy profile captured in step F) for the first exothermic reaction, and determination of the amount of energy released by the batch sample for the first exothermic reaction.

In step A), accordingly, a model calcination of the batch sample is performed. This preferably takes place under highly controlled laboratory conditions and thus under clearly reproducible conditions. This batch sample thus prepared is subsequently treated like the standard sample from the production process. Accordingly, step B) is implemented identically to step c), step C) is implemented identically to step d), step D) is implemented identically to step e), step E) is implemented identically to step f), step F) is implemented identically to step g), and step G) is implemented identically to step h). This also applies in particular to developments of the process steps of the control process that are applied correspondingly in the batch characterization process.

In step A), preparation may take place, for example, in a crucible in a muffle furnace. For a process of this kind, a period of 15 min to 1 his often required. Advantageously, however, the preparation will take place directly on a hot and heated metal on which the sample is transported simultaneously through the heating apparatus. Because of the more direct contact and the consequently improved heat transfer, substantially shorter times are possible, more particularly between 1 min and 5 min.

As a result of this, in particular, it is possible to use a first data basis for the control process in step i), before new starting materials are used, before data from the production process are available.

In a further embodiment of the invention, a first batch temperature and a first batch time selected are identical for all batch samples in step A).

In a further embodiment of the invention, the batch characterization process is repeated with a second batch temperature and a second batch time. For example, the first batch temperature is 750° C. and the second batch temperature is 850° C. In order to increase the accuracy, further batch temperatures and/or batch times are also conceivable, of course.

Below, the control process of the invention is elucidated in more detail by means of an exemplary embodiment, which is represented in the drawings.

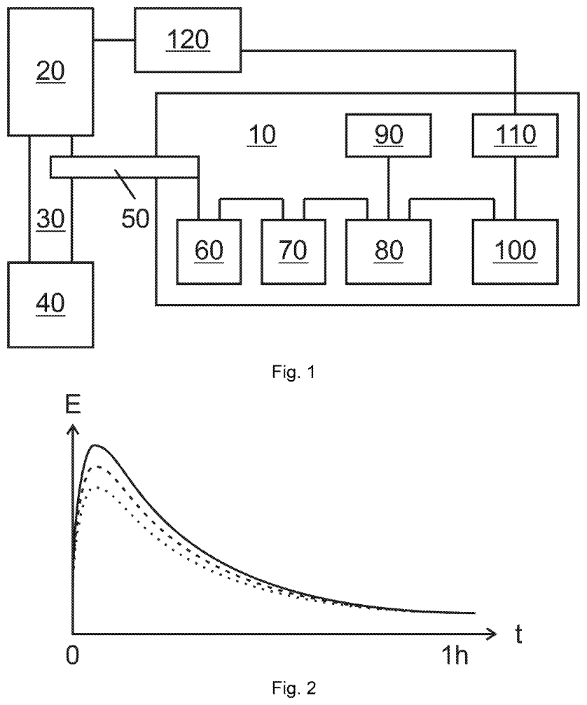

FIG. 1: Schematic representation of an apparatus for implementing the process

FIG. 2: Initial peak

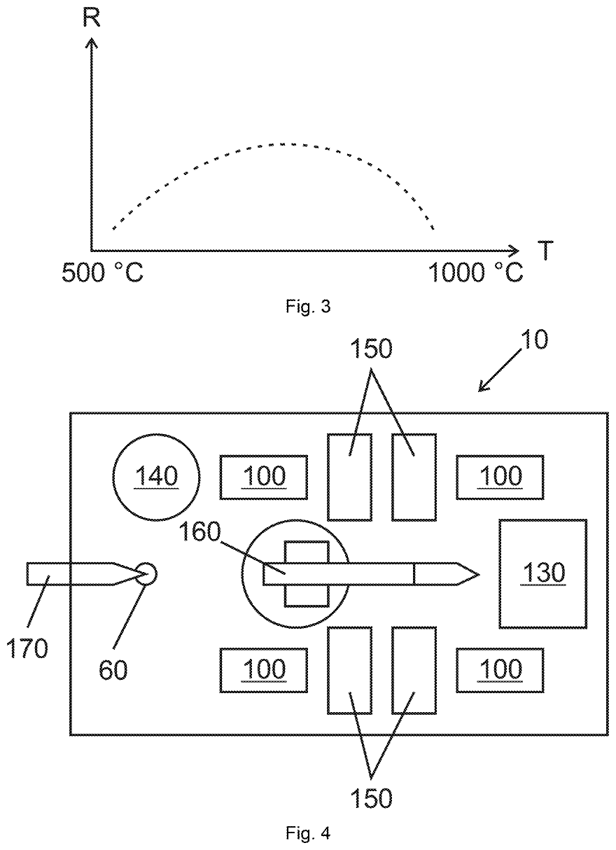

FIG. 3: Reactivity as a function of the calcination temperature

FIG. 4: Automated analytical apparatus in plan view

FIG. 1 shows an apparatus in which the control process of the invention is used. The process procedure will be elucidated with reference to the apparatus. In a calciner 20, a clay is calcined and the calcined clay is transferred via a product removal means 30 from the calciner 20 into a product store 40. In the region of the product removal means 30, a sampling means 50 takes a sample of the calcined clay and transfers the sample into an analytical apparatus 10, which has a climatized housing. For example, 21° C. is selected as first measuring temperature, and the interior of the analytical apparatus 10 is conditioned to 21° C. It has emerged that the control accuracy to an accuracy of preferably 0.1 K or better down to 0.05 K is necessary, provided no mathematical correction function is to be applied to the measured values in the event of temperature deviations. The sample is first ground using a mill 60, for example a ball mill having a sample chamber and agate grinding balls, for a predefined time, 2 min for example. In this way, a size distribution which is reproducible for all samples is established.

Subsequently, by means of a balance 70, a predefined sample mass is weighed out, of 5 g±0.02 g for example, and the sample is subsequently admixed with, for example, 10 g±0.02 g of a 1 ml/l aqueous sodium hydroxide solution and mixed briefly and intensely in a mixing apparatus. The sample-alkali mixture is then introduced into an isothermal calorimeter 100 and the energy flows resulting from the reaction are captured against the time. An analytical electronic unit 110 evaluates the overall energy of the initial peak, for the energy released in a first reaction step within the first hour. At the moment of the sample being taken by the sampling means 50, the controlling electronic unit 120 captures the temperature of the calciner 20, and correlates this information with the initial peak energy of the sample as determined by the analytical electronic unit 110 (for example, the peak maximum or integral of the peak area or the cumulative heat released at a point in time). Through comparison with previous measurements, the controlling electronic unit 120 is then able to ascertain whether a change in the temperature of the calciner 20 is useful for improving the reactivity of the calcined clay. Subsequently, either the controlling electronic unit 120 is able to drive the calciner 20 directly, by altering the fuel supply rate, for example, or the controlling electronic unit 120 is able to propose such alteration to the plant operator.

FIG. 2 shows, entirely schematically, the energy measured in an isothermal calorimeter as a function of the time for three samples produced under different production conditions. The figure shows the initial peak within the first hour of reaction for the reaction of calcined clay with three times the amount of 1 mol/l NaOH solution. The integral below the curve corresponds to the energy released in the hydrolysis and is therefore proportional to the number of reactive centers. It has emerged that the sample with continuous line shown in FIG. 2 exhibits the greatest reactivity, even for a measuring time of several days, followed by the sample with the dashed line. The sample with the dotted line has the lowest reactivity, both for the initial peak and for measurement over several days. Therefore, the area under the initial peak can be easily determined by integration over the first hour, and this area is a good direct measure of the reactivity of the sample. The maximum lies in a range from 2 min up to 10 min; after about 30 min, the measured value has already dropped below the starting value.

FIG. 3 shows a highly simplified plot of the reactivity R (proportional to the measured energy) as a function of the temperature in the calciner 20. Below 600° C., reactivity is very low; above 950° C., the reactivity drops very rapidly owing to vitrification or the partial crystallization of the product. The objective of the control process is to operate the production process as far as possible within the region of the maximum of this curve.

FIG. 4 shows an illustrative automated analytical apparatus 10 in plan view. Via a sample supply means 170, the sample is introduced, by way of a vibratory conveying channel, for example, into the analytical apparatus 10 and is preferably weighed or portioned at the same time. For this purpose, a robot 160 has taken a sample container from the sample container store 140 beforehand and has inserted it into the mill 60. A sample is introduced into the sample container and ground in the sample container in the mill 60. From there, the robot 160 transports the sample container to a space in a storage and conditioning region 150. A storage and conditioning region 150 has about 25 spaces, making a total of about 100 spaces in the example shown, at which the samples can be conditioned after grinding and before the addition of an alkali. For example and preferably, therefore, the storage and conditioning region 150 is traversed by a flow of a heat exchanger fluid, to enable extremely rapid and effective conditioning. The storage and conditioning region 150 preferably adjusts the sample to the first measuring temperature±0.1 K. Thereafter, the sample container with the sample is brought by the robot 160 into the alkali addition apparatus 130. The alkali addition apparatus 130 preferably has an alkali store 90 containing alkali conditioned to the first measuring temperature, this alkali being 1 mol/l sodium hydroxide solution, for example. The alkali is added in accordance with the exact weight of sample introduced, as for example three times the amount, i.e., 15 g of alkali to 5 g of sample. Also disposed in the alkali addition apparatus 130 is a mixing apparatus 80, preferably in the form of a compressed air feed. Moreover, the alkali addition apparatus 130 has a camera for the optical capture of the sample. This serves to recognize whether drops of alkali and sample are adhering in the upper wall region of the sample container. Should that be the case, the amount of heat released there is not reliably captured, possibly leading to measurement errors. From here, the sample holder is brought into an isothermal calorimeter 100. For example and preferably, each isothermal calorimeter has 8 measurement places. After the introduction of the sample container into the isothermal calorimeter, the measurement place is closed, preferably with two lids, by the robot 160.

### REFERENCE SIGNS

10 analytical apparatus
20 calciner
30 product removal means
40 product store
50 sampling means
60 mill
70 balance
80 mixing apparatus
90 alkali store
100 isothermal calorimeter
110 analytical electronic unit
120 controlling electronic unit
130 alkali addition apparatus
140 sample container store
150 storage and conditioning region
160 robot
170 sample supply means

1-15. (canceled)

16. A control process for controlling a production process for calcined clays with a calciner (20), comprising:
a) capture of at least one temperature in the calciner,
b) taking of at least one sample of the calcined clay,
c) production of a reproducible size distribution of the sample,
d) adjustment of the sample mass to a predefined sample mass,
e) conditioning of the sample to a first measuring temperature and of an alkali to a first measuring temperature,
f) combining of the sample with an alkali,
g) temporal capture of the energy generated by the sample-alkali mixture at constant first measuring temperature for a first period,
h) quantitative evaluation of the time-energy profile captured for the first exothermic reaction and determination of the amount of energy released by the sample for the first exothermic reaction,
i) correlation of the amount of energy captured with the temperature and residence time captured, and comparison with previously captured combinations of amount of energy, temperature and residence time values,
j) active control of the temperature and/or of the residence time in the calciner in the direction of increasing the amount of energy anticipated for a further sample,
where the quantitative evaluation of the time-energy profile captured for the first exothermic reaction takes place for the period from the first minute to minute 120.

17. The control process of claim 16, wherein the production of a reproducible size distribution of the sample takes place by grinding.

18. The control process of claim 16, wherein the alkali selected is an alkali metal hydroxide solution having a pH of between 9 and 15.

19. The control process of claim 18, wherein the mass of added alkali is 0.5 times to 5 times the mass of the sample.

20. The control process of claim 16, wherein the alkali comprises an alkali former and water, where the alkali former and the water in reaction with one another generate a solution having a pH of between 9 and 15.

21. The control process of claim 20, wherein the alkali former is selected from the group encompassing alkali metal hydroxide, alkali metal oxide, alkaline earth metal hydroxide, alkaline earth metal oxide, and substances, mixtures or compositions comprising them.

22. The control process of claim 16, wherein from production of a reproducible size distribution of the sample to temporal capture of the energy generated by the sample-alkali mixture at constant first measuring temperature for the first period take place automatically in an environment acclimatized to the first measuring temperature.

23. The control process of claim 16, wherein the sample mass is adjusted to the mandated sample mass to an accuracy of at least 2%, preferably at least 0.5%, more preferably at least 0.1%, very preferably to at least 0.02%.

24. The control process of claim 16, wherein the quantitative evaluation of the time-energy profile captured in step for the first exothermic reaction takes place for the period from the second minute up to minute 70.

25. The control process of claim 16, wherein the first measuring temperature is selected in the range from 20° C. to 40° C.

26. The control process of claim 16, wherein the predefined sample mass selected is between 1 g and 200 g, preferably between 2 g and 20 g.

27. The control process of claim 16, wherein additionally captures the reactant batches from which and the mixing ratio in which the clay is supplied to the calcination, with the information as to the reactant batches from which and the mixing ratio in which the clay has been supplied to the calcination being used additionally in step i), with control in step j) additionally considering the selection and the mixing ratio between the reactant batches.

28. The control process of claim 1627 wherein additionally to the characterization of each product batch, the following batch characterization process is carried out, with the following steps:
A) heating of a batch sample to a batch temperature of 600° C. to 1000° C., preferably 600° C. to 950° C., for a batch time of 1 s to 60 min, preferably of 30 s to 20 min, more preferably of 30 s to 5 min,
B) production of a reproducible size distribution of the batch sample,
C) adjustment of the batch sample mass to a predefined sample mass,
D) conditioning of the batch sample to a first measuring temperature,
E) combination of the batch sample with an alkali,
F) temporal capture of the energy generated by the batch sample-alkali mixture at constant first measuring temperature for a first period,
G) quantitative evaluation of the time-energy profile captured in step F) for the first exothermic reaction, and determination of the amount of energy released by the batch sample for the first exothermic reaction.

29. The control process of claim 28, wherein an identical first batch temperature and an identical first batch time are selected for all batch samples.

30. The control process of claim 28, wherein the batch characterization process is repeated with a second batch temperature and a second batch time.
